(19) United States
(12) Patent Application Publication
(10) Pub. No.: US 2024/0238123 A1
(43) Pub. Date: Jul. 18, 2024

(54) CANNULAS FOR OPHTHALMIC PROCEDURES

(71) Applicant: Alcon Inc., Fribourg (CH)

(72) Inventors: Ashish Sinha, Irvine, CA (US); Grace Chuang Liao, Irvine, CA (US)

(21) Appl. No.: 18/411,341

(22) Filed: Jan. 12, 2024

Related U.S. Application Data
(60) Provisional application No. 63/480,341, filed on Jan. 18, 2023.

(51) Int. Cl. A61F 9/007

(52) U.S. Cl. CPC A61F 9/00781

(57) ABSTRACT

Certain aspects of the present disclosure generally relate to ophthalmic surgery, and more particularly, to methods and apparatuses for controlling intraocular pressure (IOP). An exemplary apparatus generally includes a proximal segment comprising a first inner diameter (ID), an intermediate segment coupled to the proximal segment, the intermediate segment comprising a second ID smaller than the first ID, and a distal segment coupled to the intermediate segment, the distal segment comprising a third ID smaller than the second ID.

### BACKGROUND

Posterior segment surgical procedures are performed to treat conditions of the back of the eye, such as age-related macular degeneration (AMD), diabetic retinopathy and diabetic vitreous hemorrhage, macular hole, retinal detachment, epiretinal membrane, cytomegalovirus (CMV) retinitis, and others.

Certain problems affecting the back of the eye may require vitrectomy, or surgical removal of the vitreous, which is a normally clear, gel-like substance that fills the center of the eye helping to provide form and shape to the eye. For example, vitrectomy may be performed to clear blood and debris from the eye, to remove scar tissue, or to alleviate traction on the retina. During the procedure, three separate incisions are made in the pars plana of the eye, which is located just behind the iris but in front of the retina. The incisions are used to pass instruments into the eye such as a light pipe, an infusion port, and/or a vitrectomy cutting device. A valved cannula is positioned within each incision to enable instrument access via the cannula into the eye while, at the same time, providing a self-sealing valve to passively control fluid and pressure communication between inside and outside the eye via the cannula.

As vitreous fluid is aspirated during posterior segment surgery, intraocular pressure (IOP) decreases and the patient's eye tends to soften. An infusion cannula may be coupled to the valved cannulas to infuse fluid, such as liquid or gas (e.g., balanced salt solution (BSS)), to the eye to maintain IOP and avoid globe deformation or collapse. In addition, maintaining IOP may help maintain scleral rigidity to facilitate movement of the eye and exchange of instruments during the procedure. However, IOP must be carefully regulated, as prolonged periods of elevated IOP can damage eye structures. If IOP becomes too high, another infusion cannula may be used to vent fluid from the eye to relieve pressure.

Certain existing fluid cannulas (e.g., infusion cannulas, etc.) have drawbacks. For example, certain existing fluid cannulas are size specific, wherein each fluid cannula has dimensions tailored to the particular gauge of the valved cannula to which the fluid cannula is to be coupled. Valved cannulas (also referred to as valved trocar cannulas) are available in several different gauges (e.g., 23 gauge, 25 gauge, and 27 gauge), and thus a user (e.g., an ophthalmic surgeon) must have access to a fluid cannula specifically sized to the desired valved cannula gauge for a surgical procedure. Using different sized valved cannulas adds inconvenience to the procedure.

Furthermore, certain existing fluid cannula designs create relatively high fluidic friction of fluids passing therethrough. The high fluidic friction restricts overall fluid flow and requires higher pressures to maintain a given fluid flow rate. Operating at higher pressures may require larger pumps and may cause additional wear and tear on the equipment. In certain cases, due to the limitations of traditional tubing sets and viscous fluid delivery systems at high pressures, burst failure and disconnection thereof may occur. Furthermore, operating at lower pressures minimizes the risk of harm to ocular tissue.

### BRIEF SUMMARY

The present disclosure relates generally to devices, systems, and methods to control intraocular pressure during ophthalmic surgery, such as posterior segment surgical procedures including vitrectomy. More particularly, certain aspects of the present disclosure relate to an infusion cannula and methods of use thereof for infusion/venting of ocular fluids.

Certain aspects provide a cannula device for a surgical procedure, the cannula device having a proximal segment comprising a first length and a first inner diameter (ID), an intermediate segment coupled to the proximal segment, the intermediate segment comprising a second length and a second ID smaller than the first ID, and a distal segment coupled to the intermediate segment, the distal segment comprising a third length greater than the second length and a third ID smaller than the second ID.

Certain aspects provide a cannula device for a surgical procedure, the cannula device having a proximal segment comprising a first inner diameter (ID), a first length, a first proximal end, and a first distal end, an intermediate segment coupled to the proximal segment, the intermediate segment comprising a second ID smaller than the first ID, a second length, a second proximal end, and a second distal end, a first transition connecting the proximal segment and the intermediate segment via the first distal end and the second proximal end, the first transition comprising a first fillet and a second fillet, a distal segment coupled to the intermediate segment, the distal segment comprising a third ID smaller than the second ID, a third length greater than the second length, a third proximal end, and a third distal end, and a second transition connecting the intermediate segment and the distal segment via the second distal end and the third proximal end, the second transition comprising a third fillet and a fourth fillet.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure. Figures disclosed herein may not be to scale.

FIG. 1A depicts a schematic diagram of an exemplary infusion cannula assembly including an infusion cannula, in accordance with certain embodiments of the present disclosure.

FIG. 1B depicts an enlarged schematic view of a distal end of the infusion cannula assembly of FIG. 1A, in accordance with certain embodiments of the present disclosure.

FIG. 2A depicts an isometric view of an infusion cannula, in accordance with certain embodiments of the present disclosure.

FIG. 2B depicts a cross-sectional side view of the infusion cannula of FIG. 2A, in accordance with certain embodiments of the present disclosure.

FIG. 2C depicts an isometric view of the infusion cannula of FIG. 2A coupled to a valved cannula, in accordance with certain embodiments of the present disclosure.

FIG. 2D depicts a cross-sectional side view of the infusion cannula coupled to the valved cannula of FIG. 2C, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the art, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body. An intermediate segment or portion of a component refers to the segment or portion that is positioned between the distal segment or portion and the proximal end or portion.

As used herein, the term “about” may refer to a +/−10% variation from the nominal value. It is to be understood that such a variation can be included in any value provided herein.

Embodiments of the present disclosure provide devices, systems, and methods to control intraocular pressure (IOP) and/or administer fluids to the ocular space. For example, embodiments described herein disclose infusion cannulas and methods of use thereof for infusion and/or venting of ocular fluids as described in more detail below. Note that, as described herein, although the infusion cannulas are coupled to or used in conjunction with valved cannulas, certain aspects may involve use of non-valved cannula instead of valved cannulas. Also note that, although described with reference to vitreoretinal procedures, the devices, systems, and methods described herein are also applicable to other types of cannulas or uses and are not solely limited to ophthalmic procedures. Further, techniques and designs discussed herein may be similarly applicable to other types of fluid cannulas.

Certain existing fluid cannulas, including infusion cannulas, include an inner diameter (ID) that may limit fluid flow to a level that is less than what the accompanying valved cannula can support. For example, a smallest ID of certain existing fluid cannulas is smaller than a corresponding smallest ID of a valved cannula through which the fluid flows. The smallest ID of the fluid cannula restricts the fluid flow because a resistance (R) to fluid flow is directly proportional to a viscosity (η) of the fluid and a length (L) of a passage the fluid is flowing through, where the fluid passage is a portion of the fluid cannula with a constant ID. Furthermore, the resistance to fluid flow is inversely proportional to a radius of the fluid passage to the fourth power (r⁴), where the radius is one-half of the ID of the fluid passage. Thus, the resistance to fluid flow is also inversely proportional to ID of the fluid passage:

$$R ∝ \frac{η L}{{r}^{4}} \qquad Equation ( 1 )$$

Therefore, because certain existing fluid cannulas include segments having an ID smaller than the smallest ID of a corresponding valved cannula, these segments of such fluid cannulas limit fluid flow to a level that is less than the valved cannula can actually support.

Accordingly, certain embodiments described herein provide infusion cannulas that may have one or more of the following features, each of which provides a benefit.

For example, certain embodiments herein provide an infusion cannula having a distal segment with a shortened length than certain existing fluid cannulas. The shortened length of the distal segment reduces the resistance to fluid flow thereof because resistance is proportional to the length of the fluid passage.

Further, certain embodiments herein provide an infusion cannula having an intermediate segment to transition between a proximal segment and the distal segment. The intermediate segment may have a larger ID and a longer length than those in certain existing fluid cannulas. The larger ID of the intermediate segment reduces the resistance to fluid flow as the fluid passage transitions from the proximal segment to the intermediate segment. In addition, the larger ID of the intermediate segment reduces the resistance to fluid flow because it provides a larger diameter (which is inversely proportional to the resistance) for a longer portion of the length inside a compatible valved cannula. The longer length of the intermediate segment allows fluid(s) to flow a longer length/distance before transitioning into the distal segment which has the smallest ID among the three segments, thereby reducing the resistance to fluid flow.

Further, certain embodiments herein provide an infusion cannula having transitions between the segments having at least two fillets (e.g., rounds, curves, cutouts, radii, splines, or similar curvatures) and/or chamfers (e.g., bevels). The transitions provide a smooth transition between the segments. Thus, the previously described features are configured to reduce overall flow resistance and pressure drop through the infusion cannula, which, in turn, lowers the pressure needed to maintain a given fluid flow rate or, stated another way, increases the flow rate at a given source pressure, as described in more detail below.

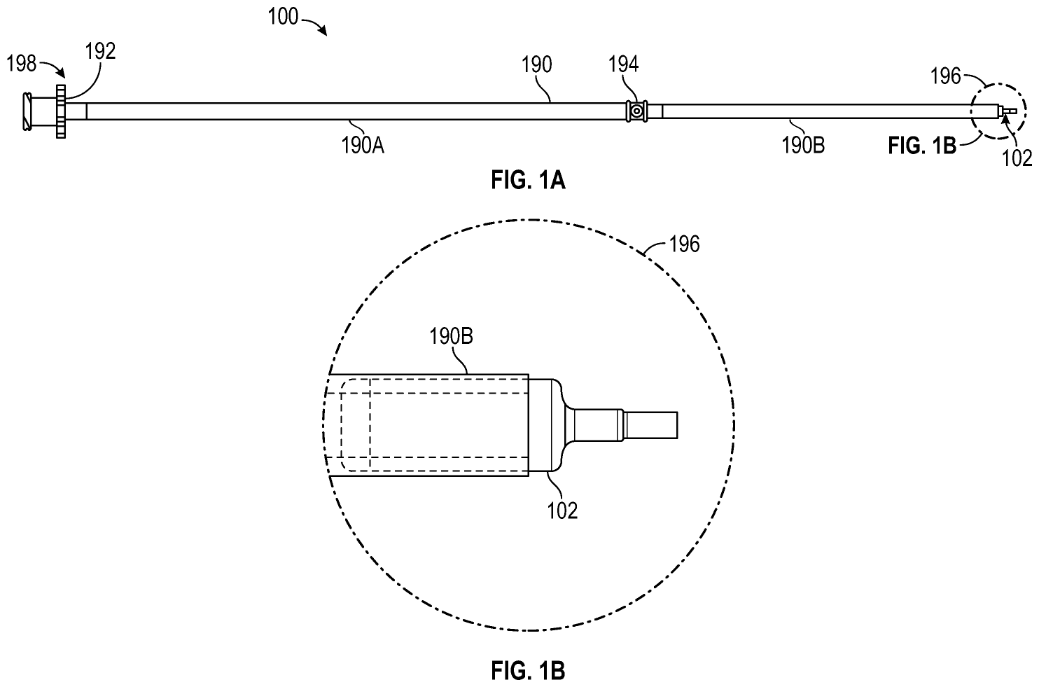

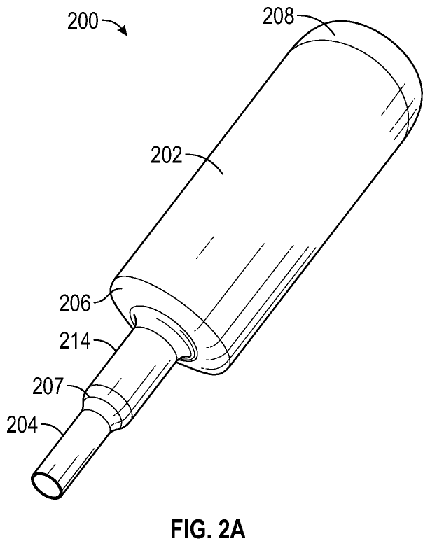

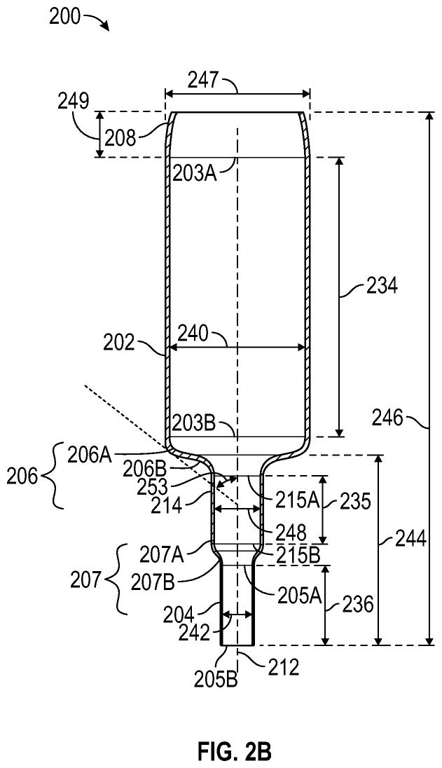

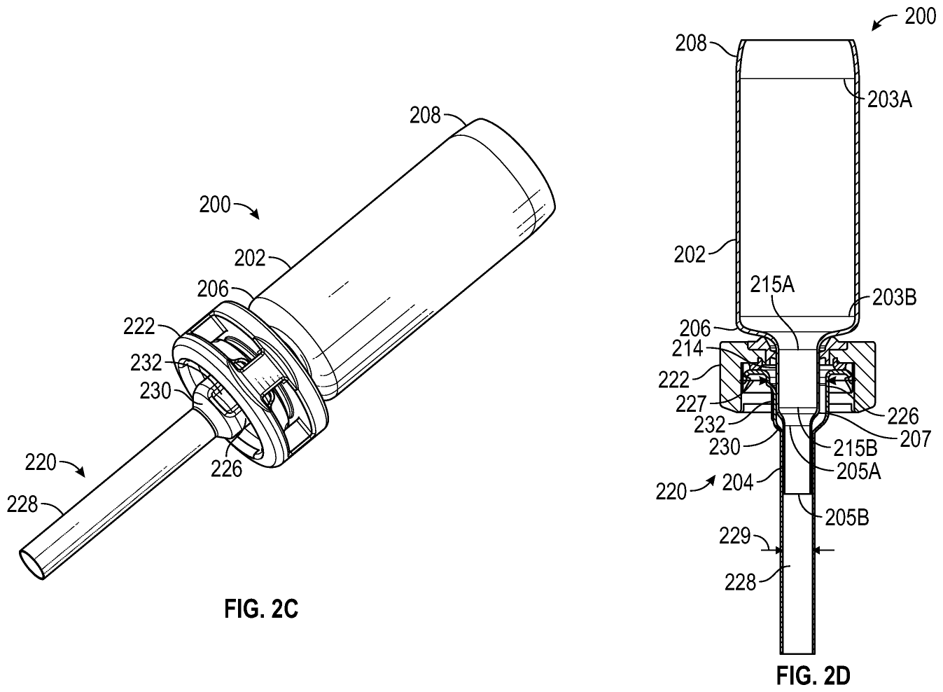

FIG. 1A depicts a schematic diagram of an exemplary infusion cannula assembly 100, according to certain embodiments. The infusion cannula assembly 100 generally includes an infusion cannula 102, an infusion tubing 190, and an adaptor 192. The infusion cannula 102 may be configured to be inserted into, e.g., a valved cannula (shown in FIGS. 2C and 2D), and may be used to move fluid through the valved cannula into the ocular space of a patient's eye.

In certain embodiments, the infusion tubing 190 may comprise two or more sections of tubing coupled by one or more connectors. As shown in FIG. 1A, the infusion tubing 190 includes a first (e.g., proximal end) infusion tubing 190A and a second (e.g., distal end) infusion tubing 190B coupled by a connector 194. In certain embodiments, the proximal end infusion tubing 190A and the distal end infusion tubing 190B may be the same type of tubing. In certain embodiments, the proximal end infusion tubing 190A and the distal end infusion tubing 190B may be different types of tubing have different features. For example, in certain embodiments, the proximal end infusion tubing 190A may comprise a plastic or polymer material, while the distal end infusion tubing 190B may comprise a metallic material. In certain embodiments, the proximal end infusion tubing 190A and distal end infusion tubing 190B may have different wall thicknesses and/or different outer diameters. In certain embodiments, the proximal end infusion tubing 190A and distal end infusion tubing 190B may have the same inner diameter, while simultaneously having different wall thicknesses.

The infusion tubing 190 comprises a fluid line that fluidically connects to the infusion cannula 102 at a distal end 196 of the infusion cannula assembly 100. The infusion tubing 190 also fluidically connects the infusion cannula 102 to a fluid source through the adaptor 192 at a proximal end 198 thereof opposite the infusion cannula 102.

In certain embodiments, the fluid source may comprise a component of a fluidic drive system, such as a fluidic drive system of a surgical console. During a surgical procedure, fluid(s) stored within the fluid source may flow through the infusion cannula assembly 100 and the valved cannula and into the patient's eye. In such embodiments, the fluidic drive system and/or surgical console may be configured to control or drive the flow of the fluid(s), based on input from a surgeon, at a set pressure and/or flow rate through the infusion tubing 190 and/or infusion cannula 102 to control the IOP of the eye. In such embodiments, the pressure and/or flow rate may be adjusted by the surgeon via actuation of a foot pedal or other mechanical or digital switch in communication with the fluidic drive system and/or surgical console.

FIG. 1B depicts an enlarged view of the distal end 196 of the infusion cannula assembly 100 having the infusion cannula 102. In particular, FIG. 1B shows a side profile of the infusion cannula 102 inserted in the infusion tubing 190.

As shown, the infusion cannula 102 connects to the infusion tubing 190, and may be made of similar or different materials thereto. In certain embodiments, the infusion cannula 102 may be made of any of a variety of materials, including metallic materials (e.g., stainless steel, carbon steel, titanium, or any suitable metallic alloy), plastic materials, rigid polymer materials (e.g., polycarbonate, polyethylene, polypropylene, polyimide), or the like. In certain embodiments, the infusion cannula 102 comprises a protective coating formed thereon, such as nickel plating, to resist corrosion and/or microbial growth. In certain embodiments, the infusion tubing 190 may be made of any of a variety of materials, including thermoplastic elastomer materials or the like.

The infusion cannula 102 and infusion tubing 190 may be manufactured using similar or different manufacturing processes. In certain embodiments, the infusion cannula 102 may be manufactured using a deep draw process or a machining process. In certain embodiments, the infusion tubing 190 may be manufactured using an injection molding process or injection overmolding process. In certain embodiments, either one or both of the infusion cannula 102 and the infusion tubing 190 may be manufactured using three-dimensional (3D) printing. In certain embodiments where the infusion cannula 102 and the infusion tubing 190 are separately manufactured, the infusion cannula 102 and the infusion tubing 190 may connect mechanically, such as through mating slots and tabs and/or friction forces, or the infusion cannula 102 and the infusion tubing 190 may attach using an adhesive or thermal bonding.

FIG. 2A depicts an isometric view of an infusion cannula 200, in accordance with certain embodiments of the present disclosure. In particular, FIG. 2A shows an exterior profile and certain exterior features of the infusion cannula 200. FIG. 2B depicts a cross-sectional side view of the infusion cannula 200 of FIG. 2A, in accordance with certain embodiments of the present disclosure. In particular, FIG. 2B shows an interior profile and certain interior features of the infusion cannula 200, and further includes several example dimensions provided in inches unless otherwise noted. In one exemplary embodiment, the infusion cannula 200 may correspond to the infusion cannula 102 in FIGS. 1A and 1B.

As shown, the infusion cannula 200 includes a proximal segment 202, an intermediate segment 214, and a distal segment 204. The proximal segment 202 includes a first proximal end 203A and a first distal end 203B; the intermediate segment 214 includes a second proximal end 215A and second distal end 215B; and the distal segment 204 includes a third proximal end 205A and third distal end 205B. A first transition 206 connects the proximal segment 202 and the intermediate segment 214 at the first distal end 203B and the second proximal end 215A. A second transition 207 connects the intermediate segment 214 and the distal segment 204 at the second distal end 215B and the third proximal end 205A. A third transition 208 is configured to facilitate connection of the proximal segment 202 and a tubing of an infusion line (e.g., the infusion tubing 190 in FIGS. 1A and 1B) at the first proximal end 203A.

In certain embodiments, the third transition 208 may have a length 249. In certain embodiments, the length 249 of the third transition 208 is between about 0.0247 inches (about 0.6261 mm (millimeters)) and about 0.0334 inches (about 0.8471 mm), such as between about 0.0261 inches (about 0.6629 mm) and about 0.0319 inches (about 0.8103 mm), such as about 0.029 inches (about 0.7366 mm). In certain embodiments, the infusion cannula 200 may not include the third transition 208. In such embodiments, the infusion cannula 200 has a total length that extends from the first proximal end 203A to the third distal end 205B of the distal segment 204. In certain embodiments, the length 249 of the third transition 208 may be greater or less than 0.029 inches (0.7366 mm).

As shown, the proximal segment 202 has a first ID 240 and a first length 234; the intermediate segment 214 has a second ID 248 and a second length 235; and the distal segment 204 has a third ID 242 and a third length 236. In certain embodiments, the proximal segment 202, intermediate segment 214, and distal segment 204 may each have a substantially uniform diameter along their respective lengths 234, 235, and 236.

In certain embodiments, the proximal segment 202 has inner dimensions, e.g., the first ID 240. In certain embodiments, the first ID 240 is between about 0.0723 inches (about 1.8352 mm) and about 0.0978 inches (about 2.4829 mm), such as between about 0.0765 inches (about 1.9431 mm) and about 0.0935 inches (about 2.3749 mm), such as about 0.085 inches (about 2.159 mm). In certain embodiments, the first ID 240 of the proximal segment 202 is greater than 0.085 inches (2.159 mm), such as when an exterior of the proximal segment 202 conforms to the inside of the tubing of the infusion line (e.g., the infusion tubing 190 in FIG. 1). In other embodiments, the first ID 240 of the proximal segment 202 may be less than 0.085 inches (2.159 mm).

In certain embodiments, the intermediate segment 214 has inner dimensions, e.g., the second ID 248. In certain embodiments, the second ID 248 is between about 0.0247 inches (about 0.6261 mm) and about 0.0334 inches (about 0.8471 mm), such as between about 0.0261 inches (about 0.6629 mm) and about 0.0319 inches (about 0.8103 mm), such as about 0.029 inches (about 0.7366 mm). In certain embodiments, the second ID 248 of the intermediate segment 214 is greater than half of the first ID 240 of the proximal segment 202. In certain embodiments, the second ID 248 of the intermediate segment 214 is greater than 0.029 inches (0.7366 mm), such as when an exterior of the intermediate segment 214 conforms to the inside of a valved cannula hub (e.g., hub 226 in FIG. 2D) and/or a cannula transition (e.g., a cannula transition 230 in FIG. 2D), or when the distal segment 204 has an indentation. In other embodiments, the second ID 248 of the intermediate segment 214 may be less than 0.029 inches (0.7366 mm).

In certain embodiments, the distal segment 204 has inner dimensions, e.g., the third ID 242, of between about 0.0151 inches (about 0.3835 mm) and about 0.0155 inches (about 0.3937 mm), such as about 0.0153 inches (about 0.3886 mm) (e.g., about 27 gauge). In certain embodiments, the third ID 242 may be between about 0.0191 inches (about 0.4851 mm) and about 0.0195 inches (about 0.4953 mm), such as about 0.0193 inches (about 0.4902 mm) (e.g., about 25 gauge). In certain embodiments, the third ID 242 may be between about 0.0240 (about 0.6096 mm) and about 0.0244 inches (about 0.6198 mm), such as about 0.0242 inches (about 0.6147 mm) (e.g., about 23 gauge). In certain embodiments, the inner dimensions of the distal segment 204 are greater than 0.0244 inches (0.6198 mm), such as when an exterior of the distal segment 204 conforms to the inside of the shaft of a valved cannula hub (e.g., a shaft 228 in FIG. 2D) and/or a cannula transition (e.g., the cannula transition 230 in FIG. 2D), or when the distal segment 204 has an indentation.

In certain embodiments, the infusion cannula 200 has a total width 247, which may be a diameter between about 0.0765 inches (about 1.9431 mm) and about 0.1035 inches (about 2.6289 mm), such as between about 0.081 inches (about 2.0574 mm) and about 0.099 inches (about 2.5146 mm), such as about 0.09 inches (about 2.286 mm). In certain embodiments, the total width 247 of the infusion cannula 200 may be greater or less than 0.09 inches (2.286 mm).

Generally, the segments 202, 204, and 214 and the transitions 206, 207, and 208 share a common central axis 212 disposed through a common center of the infusion cannula 200, which may be formed from a single, unitary body. Note that although described with the segments 202, 204, and 214 and the transitions 206, 207, and 208, the infusion cannula 200 may be formed from a single, unitary body and the segments 202, 204, and 214 and the transitions 206, 207, and 208 may refer to portions of the unitary body.

In certain embodiments, the proximal segment 202 has a first length 234, the intermediate segment 214 has a second length 235, and the distal segment 204 has a third length 236, each measured along the central axis 212. The second length 235 may be more than, less than, or equal to the first length 234. The third length 236 may be more than, less than, or equal to the second length 235. In further embodiments, the third length 236 is substantially less (or shorter) than the second length 235. In further embodiments, the third length 236 is substantially zero, and the infusion cannula 200 has a total length extending between the third transition 208 and a distal end of the second transition 207 (e.g., the infusion cannula 200 does not include the distal segment 204).

In certain embodiments, the first length 234 of the proximal segment 202 is between about 0.1496 inches (about 3.7998 mm) and about 0.2024 inches (about 5.1410 mm), such as between about 0.1584 inches (about 4.0234 mm) and about 0.1936 inches (about 4.9174 mm), such as about 0.176 inches (about 4.4704 mm). In certain embodiments, the first length 234 of the proximal segment 202 is greater than 0.176 inches (4.4704 mm), such as when an exterior of the proximal segment 202 conforms to the inside of the tubing of the infusion line (e.g., the infusion tubing 190 in FIG. 1). In other embodiments, the first length 234 of the proximal segment 202 may be less than 0.176 inches (4.4704 mm).

In certain embodiments, the second length 235 of the intermediate segment 214 is between about 0.034 inches (about 0.8636 mm) and about 0.046 inches (about 1.1684 mm), such as between about 0.036 inches (about 0.9144 mm) and about 0.044 inches (about 1.1176 mm), such as about 0.04 inches (about 1.016 mm). In certain embodiments, the second length 235 of the intermediate segment 214 is greater than 0.04 inches (1.016 mm), such as when an exterior of the intermediate segment 214 conforms to the inside of a valved cannula hub (e.g., the hub 226 in FIG. 2D) and/or a cannula transition (e.g., the cannula transition 230 in FIG. 2D), or when the distal segment 204 has an indentation. In other embodiments, the second length 235 of the intermediate segment 214 may be less than 0.04 inches (1.016 mm).

In certain embodiments, the second ID 248 and the second length 235 is larger than those in certain existing fluid cannulas. The larger ID of the intermediate segment 214 reduces the resistance to fluid flow as the fluid passage transitions from the proximal segment 202 to the intermediate segment 214. In addition, the larger ID of the intermediate segment 214 reduces the resistance to fluid flow because it provides a larger diameter (which is inversely proportional to the resistance) for a portion of the length inside a compatible valved cannula (e.g., the valved cannula 220 in FIG. 2C). The longer length (e.g., the second length 235) of the intermediate segment allows fluid(s) to flow through a longer length/distance before transitioning into the distal segment 204, which has the smallest ID among the three segments, thereby reducing the resistance to fluid flow. For example, in certain embodiments, the flow rate is increased by about 5% to about 30% or greater as compared to certain existing cannula designs. In some examples, the flow rate may be increased by more than 30%, such as between 30% and 35% or greater than certain existing cannula designs. In certain embodiments, the pressure drop is decreased proportionally, e.g., by about 20% to about 35% or greater. As described above, the flow rate and pressure drop are affected by the ID and length of passage of the segments, as well as the source pressure and fluid viscosity.

In certain embodiments, the third length 236 of the distal segment 204 is between about 0.0425 inches (about 1.0795 mm) and about 0.0575 inches (about 1.4605 mm), such as between about 0.045 inches (about 1.143 mm) and about 0.055 inches (about 1.397 mm), such as about 0.05 inches (about 1.27 mm). In certain embodiments, the third length 236 of the distal segment 204 is greater than 0.05 inches (1.27 mm), such as when an exterior of the distal segment 204 conforms to the inside of the shaft of the valved cannula hub (e.g., the shaft 228 in FIG. 2D) and/or the cannula transition (e.g., the cannula transition 230 in FIG. 2D), or when the distal segment 204 has an indentation. In certain embodiments, the third length 236 of the distal segment 204 is shorter than the second length 235 of the intermediate segment 214. In other embodiments, the third length 236 of the distal segment 204 may be less than 0.05 inches (1.27 mm).

Furthermore, in certain embodiments, the distal segment 204 is shorter than certain existing fluid cannula designs because the extended distal segment of such infusion cannulas is a limiting factor for fluid flow. The flow rate is generally affected by the input pressure and viscosity of fluids being flowed through the infusion cannula 200. The flow rate is further affected by the ID of the segments, because narrowing the cross section increases the pressure loss, leading to a decreased flow rate. Shortening the distal segment 204 reduces the distance the fluid flows through the smallest ID (e.g., the third ID 242) and prevents an excessive pressure loss by providing a longer, wider cross section of the shaft 228 for the fluid to flow. In certain embodiments, the shortened length of the distal segment 204 reduces the resistance to fluid flow thereof because it minimizes the length of the passage the fluid(s) flowing through the segment (e.g., the distal segment 204) of the infusion cannula 200 with the smallest ID. For example, the shorter distal segment 204 may increase the flow rate about 5-30% or greater compared to certain existing fluid cannula designs. In some examples, the flow rate may be increased by more than 30%, such as between 30% and 35% or greater.

In certain embodiments, the relatively larger ID (e.g., the second ID 248) and longer length (e.g., the second length 235) of the intermediate segment 214 in combination with the shortened length (e.g., the third length 236) of the distal segment 204 reduce an overall source pressure required to maintain a given flow rate with the infusion cannula 200 disclosed herein. In certain embodiments, the source pressure is reduced proportional to the decrease in flow resistance. In certain embodiments, the flow rate is increased at a given source pressure with injection cannula embodiments disclosed herein. In certain embodiments, the increase in the flow rate is generally about 5-30% or more. In certain embodiments, such as at 80 psi (pounds per square inch) source pressure, the increase in the flow rate is about 15-30% or greater as compared to certain existing 25 gauge injection cannula designs. In certain embodiments, such as at 30 psi source pressure, the increase in the flow rate is about 5-20% or greater compared to certain existing 25 gauge injection cannula designs.

In certain embodiments, the infusion cannula 200 has an internal shaft length 244 (e.g., the distal segment 204, the transition 207, the intermediate segment 214, and the second fillet 206B) of between about 0.1003 inches (about 2.5476 mm) and about 0.1357 inches (about 3.4468 mm), such as between about 0.1062 inches (about 2.6975 mm) and about 0.1298 inches (about 3.2969 mm), such as about 0.118 inches (about 2.9972 mm). In other embodiments, the internal shaft length 244 of the infusion cannula 200 may be greater or less than 0.118 inches (2.9972 mm).

In certain embodiments, the infusion cannula 200 has a total length 246 of between about 0.2831 inches (about 7.1895 mm) and about 0.3830 inches (about 9.7269 mm), such as between about 0.2997 inches (about 7.6124 mm) and about 0.3663 inches (about 9.3040 mm), such as about 0.333 inches (about 8.4582 mm). In other embodiments, the total length 246 of the infusion cannula 200 may be greater or less than 0.333 inches (8.4582 mm).

Note that although certain features are described herein with particular dimensions or ranges of dimensions, other dimensions and/or IDs are further contemplated.

In certain embodiments, the first transition 206 includes a first fillet 206A and a second fillet 206B, and the second transition 207 includes a third fillet 207A and a fourth fillet 207B. In certain embodiments, the first fillet 206A is concave and the second fillet 206B is convex. In certain embodiments, the third fillet 207A is concave and the fourth fillet 207B is convex.

The proximal segment 202 and the intermediate segment 214 are connected by a transition having the fillets 206A and 206B such that the flow path experiences a gradual reduction in ID when transitioning between the cylindrical shape of the proximal segment 202 and the cylindrical shape of the intermediate segment 214. The gradual reduction in ID reduces the flow resistance therethrough, when compared to a sudden reduction in ID in a configuration where a proximal segment having a relatively wide ID transitions to an intermediate segment having a relatively narrow ID.

In certain embodiments, the first transition 206 of the infusion cannula 200 may include a first chamfer or bevel disposed between the fillets 206A and 206B. In certain embodiments, a transition angle θ (e.g., a transition angle) measures between the first chamfer and an axis of one of the segments 202 and 214, which may not be collinear with the central axis 212. In certain embodiments, the transition angle θ measures between the first chamfer and the central axis 212. In certain embodiments, the transition angle θ is about 60 degrees, but in further embodiments may be about 15 degrees and up to and including about 90 degrees. In further embodiments, the transition angle θ is more than 90 degrees.

The morphology of the first transition 206 may improve the flow of fluid through the infusion cannula 200. For example, the first transition 206 includes the first chamfer which forms a funnel-shaped feature in the infusion cannula 200 having a gradual reduction in ID between the proximal segment 202 and the intermediate segment 214. The first chamfer may facilitate prevention of excessive pressure loss when compared to the transition of certain existing infusion cannulas by providing a gradual, linearly decreasing reduction in the cross section for the fluid. In certain embodiments, the first transition 206 may include the first chamfer or bevel rather than a fillet (e.g., fillets 206A and 206B).

The intermediate segment 214 and distal segment 204 are connected by a transition having the fillets 207A and 207B such that the flow path experiences a gradual reduction in ID when transitioning between the cylindrical shape of the intermediate segment 214 and the cylindrical shape of the distal segment 204. The gradual reduction in ID reduces the flow resistance therethrough, when compared to a sudden reduction in ID in a configuration where an intermediate segment having a wide ID transitions to a distal segment having a narrow ID.

In certain embodiments, the third distal end 205B of the distal segment 204 may include a sixth fillet which may be convex and/or a second chamfer or bevel. The sixth fillet and/or the second chamfer may beneficially facilitate the insertion of the infusion cannula 200 into a valved cannula 220 (e.g., in FIG. 2C) by gradually increasing the outer diameter of the third distal end 205B.

As shown in FIG. 2B, the fillets 206A, 206B, 207A, and 207B are formed on the inner walls of the transitions 206, 207, and 208, respectively. In certain embodiments, the fillets are formed on inner and/or outer walls of the transitions 206, 207, and 208, respectively. In certain embodiments, inner and/or outer walls of the segments 202, 204, and 214 are parallel or tapered relative to the central axis 212. In some embodiments, the wall thicknesses of the segments 202, 204, and 214 and the transitions 206, 207, and 208 are constant. In certain embodiments, the wall thicknesses may be variable or may vary by feature (e.g., the first transition 206, the distal segment 204).

Fillets described herein, including fillets 206A and 206B, may have one or more positional angles (e.g., positional angle 253) to reference the location of a midpoint of the fillet relative to the central axis 212. The positional angles may measure between the central axis 212 and a tangential line of the midpoint of the fillet. In certain embodiments, the positional angles may be between 1 and 85 degrees. In certain embodiments, the positional angles may be between 10 and 60 degrees. In certain embodiments, the positional angles may be between 20 and 40 degrees. For example, the positional angle 253 may be about 75 degrees. In certain embodiments, the fillet may be on an inner surface of the transition (e.g., the first transition 206). In further embodiments, the fillet may be on an outer surface of the transition.

In certain embodiments, a continuous flow path is formed between the first proximal end 203A and the third distal end 205B of the infusion cannula 200. Accordingly, curvatures of the transitions 206, 207, and 208 may allow a fluid to smoothly flow through the infusion cannula 200 by reducing fluid friction losses, thereby beneficially improving fluidic performance. In certain embodiments, the inner walls of the segments 202, 214, and 204 and the transitions 206, 207, and 208 connect seamlessly to form a smooth inner profile such that there are no disjointed transitions, interruptions, or indications of disparity between the segments 202, 214, and 204 and the transitions 206, 207, and 208.

As shown in FIG. 2A, the infusion cannula 200 has a unitary body formed of three cylinders of substantially uniform diameters: a wide proximal cylinder (e.g., the proximal segment 202), a middle intermediate cylinder (e.g., the intermediate segment 214), and a narrow distal cylinder (e.g., the distal segment 204). The middle intermediate and wide proximal cylinders are connected by a transition consisting of two fillets such that the flow path experiences a gradual reduction in ID when transitioning from the wide proximal cylinder to the middle intermediate cylinder to avoid an increase in flow resistance that would otherwise have been resulted due to a sudden reduction in ID between any two segments.

FIG. 2C depicts an isometric view of the infusion cannula 200 in FIG. 2A coupled to a valved cannula 220, in accordance with certain embodiments of the present disclosure. In particular, FIG. 2C shows an exterior profile and certain exterior features of the infusion cannula 200 and the valved cannula 220. FIG. 2D depicts a cross-sectional side view of the infusion cannula 200 coupled to the valved cannula 220 in FIG. 2C, in accordance with certain embodiments of the present disclosure. In particular, FIG. 2D shows an interior profile and certain interior features of the infusion cannula 200 coupled to the valved cannula 220. In certain embodiments, the infusion cannula 200 is configured to be inserted into the valved cannula 220 to infuse fluid through the valved cannula 220 and into the ocular space of a patient's eye. In certain embodiments, the valved cannula 220 is a valved trocar cannula.

The valved cannula 220 generally includes an overcap 222, a hub 226, and a hollow tube or shaft 228. The hub 226 and the shaft 228 are connected by the cannula transition 230. An inner diameter (ID) (e.g., an ID 227 in FIG. 2D) of the hub 226 is larger than an ID (e.g., an ID 229 in FIG. 2D) of the shaft 228. The valved cannula 220 further includes an indentation 232, which may be part of the hub 226 and/or the cannula transition 230.

In certain embodiments, at least a portion of the infusion cannula 200 frictionally engages with an inner feature of the valved cannula 220 when the infusion cannula 200 is inserted therein. For example, at least a portion of the intermediate segment 214 frictionally engages with the inner feature of the valved cannula 220, and/or at least a portion of the distal segment 204 frictionally engages with an inside of the shaft 228, resulting in a “tube in tube” configuration between the distal segment 204 and the shaft 228. The tube in tube configuration results in a stable fluid flow and operating pressure when using the infusion cannula 200. In certain embodiments, the distal segment 204 of the infusion cannula 200 is sized to closely fit a specific gauge size of the valved cannula 220. For example, the distal segment 204 has an outer diameter configured to closely fit within the shaft 228 of a 23 gauge valved cannula, a 25 gauge valved cannula, a 27 gauge valved cannula, or the like.

Different segments (e.g., the segments 214 and 204) may be passed through different portions (e.g., the hub 226 or the shaft 228) of the valved cannula 220 when the infusion cannula 200 is coupled thereto. For example, when the infusion cannula 200 and valved cannula 220 are connected, the intermediate segment 214 of the infusion cannula 200 may be disposed within the hub 226 of the valved cannula 220 while the distal segment 204 is disposed within the shaft 228.

As further shown in FIG. 2D, the hub 226 of the valved cannula 220 radially surrounds and couples to the intermediate segment 214 of the infusion cannula 200 when the infusion cannula 200 is inserted into the valved cannula 220. In certain embodiments, when inserted into the valved cannula 220, the intermediate segment 214 is disposed only inside (i.e., radially inward of) the hub 226, or disposed only inside the hub 226 and the cannula transition 230, and the intermediate segment 214 does not extend into the shaft 228. Because the intermediate segment 214 does not extend into the shaft 228, the dimensions of the intermediate segment 214 are not restricted by the dimensions of the shaft 228, and in certain embodiments, a smallest ID (e.g., the third ID 242 in FIG. 2D) of the distal segment 204 is greater than the ID 229 of the shaft 228 of the valved cannula 220. As previously described, the valved cannula 220 may be a 23 gauge valved cannula, a 25 gauge valved cannula, a 27 gauge valved cannula, and the like. The relatively greater ID (e.g., the second ID 248 in FIG. 2B) of the intermediate segment 214 compared to the ID 229 of the shaft 228 is configured to reduce overall flow resistance and pressure drop through the infusion cannula 200 because the valved cannula 220 limits fluid flow to a level that is less than what the infusion cannula 200 can actually support.

The intermediate segment 214 further enables a universal fit for the infusion cannula 200 with valved cannulas of different gauges, since the intermediate segment 214 is not required to conform to the different IDs (e.g., the ID 229) of the shafts (e.g., the shaft 228) thereof. For example, the infusion cannula 200 may universally couple to a 23 gauge valved cannula, 25 gauge valved cannula, 27 gauge valved cannula, and the like. Accordingly, the versatility of the infusion cannula 200 beneficially reduces the number of different parts needed for a surgical procedure.

In certain embodiments, the proximal segment 202 is coupled to the intermediate segment 214 through the fillets 206A and 206B such that a surface between the fillets 206A and 206B is configured to be substantially flush with the top surface of the overcap 222 when the infusion cannula 200 is fully inserted into the valved cannula 220. As compared to infusion cannulas with a tampered transition between the proximal segment and the intermediate or distal segment, the fillets 206A and 206B may provide a visual cue to the surgeon confirming when the infusion cannula 200 is fully inserted into the valved cannula 220.

In certain embodiments, the infusion cannula 200 may also include a retention feature to frictionally engage with the inner feature of the valved cannula 220 in certain embodiments. For example, the intermediate segment 214 or the distal segment 204 may have an indentation configured to mate with, closely fit, and/or conform to the indentation 232.

In certain embodiments, the indentation 232 is dimensioned to provide enough resistance between the infusion cannula 200 and the valved cannula 220 to keep the infusion cannula 200 in place during a procedure. In certain embodiments, the resistance between the indentation 232 and the infusion cannula 200 is less than needed to pull the valved cannula 220 out of the eye when withdrawing the infusion cannula 200 from the valved cannula 220. For example, the valved cannula 220 is not pulled out of the eye when the infusion cannula 200 is pulled out of the valved cannula 220 while the valved cannula 220 is in the eye. In certain embodiments, the resistance between the infusion cannula 200 and the valved cannula 220 is such that the infusion cannula 200 cannot be decoupled from the valved cannula 220 without removing the valved cannula 220 from the eye. In certain embodiments, the resistance is formed between other areas or sections of the infusion cannula 200 and the valved cannula 220. For example, in some embodiments, the exterior of the distal segment 204 frictionally engages with the interior surface of the hub 226 and/or cannula transition 230.

In summary, embodiments of the present disclosure include fluid cannulas for improved fluid administration and fluid flow during ophthalmic surgical procedures. For example, embodiments described herein provide efficient administration of ocular infusion fluids and tamponades, thereby facilitating improved intraocular pressure maintenance. Accordingly, the aforementioned cannulas are particularly beneficial during infusion of fluids to the eye, as fluidic resistance is decreased, thus enabling less operating pressure to achieve a given flow rate.

### EXAMPLE EMBODIMENTS

Embodiment 1: A cannula device for a surgical procedure comprising a proximal segment, the proximal segment comprising a first length and a first inner diameter (ID), an intermediate segment coupled to the proximal segment, the intermediate segment comprising a second length and a second ID smaller than the first ID, and a distal segment coupled to the intermediate segment, the distal segment comprising a third length smaller than the second length and a third ID smaller than the second ID.

Embodiment 2: The cannula device of Embodiment 1 described above, wherein the second ID of the intermediate segment is greater than half of the first ID of the proximal segment.

Embodiment 3: The cannula device of Embodiment 1 described above, wherein at least one portion of the distal segment is configured to be disposed inside a shaft of a valved cannula comprising a hub, the shaft, and a cannula transition between the hub and the shaft.

Embodiment 4: The cannula device of Embodiment 3 described above, wherein the at least one portion of the distal segment is configured to frictionally engage with an inner surface of the shaft of the valved cannula.

Embodiment 5: The cannula device of Embodiment 1 described above, wherein at least one portion of the intermediate segment is configured to be disposed inside a hub or a cannula transition of a valved cannula comprising the hub, a shaft, and the cannula transition between the hub and the shaft.

Embodiment 6: The cannula device of Embodiment 5 described above, wherein the at least one portion of the intermediate segment is configured to frictionally engage with an inner surface of the hub or the cannula transition of the valved cannula.

Embodiment 7: The cannula device of Embodiment 1 described above, further comprises a first transition connecting a distal end of the proximal segment and a proximal end of the intermediate segment, the first transition comprising a first fillet and a second fillet.

Embodiment 8: The cannula device of Embodiment 7 described above, further comprising a second transition connecting a distal end of the intermediate segment and a proximal end of the distal segment, the second transition comprising a third fillet and a fourth fillet.

Embodiment 9: A cannula device for a surgical procedure comprising a proximal segment comprising a first inner diameter (ID), a first length, a first proximal end, and a first distal end, an intermediate segment coupled to the proximal segment, the intermediate segment comprising a second ID smaller than the first ID, a second length, a second proximal end, and a second distal end, a first transition connecting the proximal segment and the intermediate segment via the first distal end and the second proximal end, the first transition comprising a first fillet and a second fillet, a distal segment coupled to the intermediate segment, the distal segment comprising a third ID smaller than the second ID, a third length greater than the second length, a third proximal end, and a third distal end, and a second transition connecting the intermediate segment and the distal segment via the second distal end and the third proximal end, the second transition comprising a third fillet and a fourth fillet.

Embodiment 10: The cannula device of Embodiment 9 described above, wherein the second ID of the intermediate segment is greater than half of the first ID of the proximal segment.

Embodiment 11: The cannula device of Embodiment 9 described above, wherein at least one portion of the distal segment is configured to be disposed inside a shaft of a valved cannula comprising a hub, the shaft, and a cannula transition between the hub and the shaft, and wherein at least one portion of the intermediate segment is configured to be disposed inside the hub or the cannula transition of the valved cannula.

The preceding description is provided to enable any person skilled in the art to practice the various embodiments described herein. The examples described herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A cannula device for a surgical procedure, the cannula device comprising:
a proximal segment comprising a first length and a first inner diameter (ID);
an intermediate segment coupled to the proximal segment, the intermediate segment comprising a second length and a second ID smaller than the first ID; and
a distal segment coupled to the intermediate segment, the distal segment comprising a third length greater than the second length and a third ID smaller than the second ID.

2. The cannula device of claim 1, wherein the second ID of the intermediate segment is greater than half of the first ID of the proximal segment.

3. The cannula device of claim 1, wherein at least one portion of the distal segment is configured to be disposed inside a shaft of a valved cannula comprising a hub, the shaft, and a cannula transition between the hub and the shaft.

4. The cannula device of claim 3, wherein the at least one portion of the distal segment is configured to frictionally engage with an inner surface of the shaft of the valved cannula.

5. The cannula device of claim 1, wherein at least one portion of the intermediate segment is configured to be disposed inside a hub or a cannula transition of a valved cannula comprising the hub, a shaft, and the cannula transition between the hub and the shaft.

6. The cannula device of claim 5, wherein the at least one portion of the intermediate segment is configured to frictionally engage with an inner surface of the hub or the cannula transition of the valved cannula.

7. The cannula device of claim 1, further comprising a first transition connecting a distal end of the proximal segment and a proximal end of the intermediate segment, the first transition comprising a first fillet and a second fillet.

8. The cannula device of claim 7, further comprising a second transition connecting a distal end of the intermediate segment and a proximal end of the distal segment, the second transition comprising a third fillet and a fourth fillet.

9. The cannula device of claim 8, wherein the third length of the distal segment is shorter than a combined length of the intermediate segment and the second transition.

10. A cannula device for a surgical procedure, the cannula device comprising:
a proximal segment comprising a first inner diameter (ID), a first length, a first proximal end, and a first distal end;
an intermediate segment coupled to the proximal segment, the intermediate segment comprising a second ID smaller than the first ID, a second length, a second proximal end, and a second distal end;
a first transition connecting the proximal segment and the intermediate segment via the first distal end and the second proximal end, the first transition comprising a first fillet and a second fillet;
a distal segment coupled to the intermediate segment, the distal segment comprising a third ID smaller than the second ID, a third length greater than the second length, a third proximal end, and a third distal end; and
a second transition connecting the intermediate segment and the distal segment via the second distal end and the third proximal end, the second transition comprising a third fillet and a fourth fillet.

11. The cannula device of claim 10, wherein the second ID of the intermediate segment is greater than half of the first ID of the proximal segment.

12. The cannula device of claim 10, wherein the third length of the distal segment is shorter than a combined length of the intermediate segment and the second transition.

13. The cannula device of claim 10, wherein at least one portion of the distal segment is configured to be disposed inside a shaft of a valved cannula comprising a hub, the shaft, and a cannula transition between the hub and the shaft.

14. The cannula device of claim 13, wherein the at least one portion of the distal segment is configured to frictionally engage with an inner surface of the shaft of the valved cannula.

15. The cannula device of claim 10, wherein at least one portion of the intermediate segment is configured to be disposed inside a hub or a cannula transition of a valved cannula comprising the hub, a shaft, and the cannula transition between the hub and the shaft, and wherein the at least one portion of the intermediate segment is configured to frictionally engage with an inner surface of the hub or the cannula transition of the valved cannula.
